(19) United States
(12) Patent Application Publication
(10) Pub. No.: US 2019/0083119 A1
(43) Pub. Date: Mar. 21, 2019

(54) TREATMENT TOOL

(71) Applicant: Olympus Corporation, Tokyo (JP)

(72) Inventor: Ojiro Kitamura, Tokyo (JP)

(73) Assignee: Olympus Corporation, Tokyo (JP)

(21) Appl. No.: 16/196,977

(22) Filed: Nov. 20, 2018

Related U.S. Application Data
(63) Continuation of application No. PCT/JP2016/066261, filed on Jun. 1, 2016.

(51) Int. Cl. A61B 17/29; A61B 18/14

(52) U.S. Cl. CPC A61B 17/29; A61B 2017/2926; A61B 2017/2902; A61B 18/1442

(57) ABSTRACT

A treatment tool includes a rotary member that is rotatable in unison with an elongated member such as a sheath or shaft and an end effector around a predetermined axis of rotation with respect to a housing. The rotary member has a ridge projecting toward an outer circumferential side and extending from a proximal-end side toward a distal-end side. The treatment tool includes a cavity that is concave toward an inner circumferential side. The cavity is contiguous to the distal-end side of the ridge on the rotary member or is disposed on the distal-end side from the ridge on the housing.

### CROSS-REFERENCE TO RELATED APPLICATION

This application is a continuation application of PCT Application No. PCT/JP2016/066261 filed on Jun. 1, 2016, which is hereby incorporated by reference in its entirety.

### TECHNICAL FIELD

The disclosed technology relates to a treatment tool for treating a treatment target with an end effector.

### DESCRIPTION OF THE RELATED ART

U.S. Pat. No. 5,383,888 discloses a treatment tool having an end effector for treating a treatment target. The end effector is disposed on the distal end of a shaft. The shaft is coupled to a portable housing. When a handle of the treatment tool is closed or opened with respect to a grip of the handle, the space between a pair of gripping members of the end effector is closed or opened accordingly. When the space between the gripping members is closed, a treatment target such as a living tissue or the like is gripped between the gripping members. A rotary member or rotary knob is mounted on the housing such that the rotary member is rotatable about the central axis of the shaft. When a user applies a manipulating force for rotating the rotary member, the shaft and the end effector are rotated about the central axis of the shaft as a predetermined axis of rotation in unison with the rotary member with respect to the housing. The angular position of the end effector about the predetermined axis of rotation is thus changed. Furthermore, the end effector is bent with respect to the shaft, i.e., the central axis of the shaft, based on a manipulation of a bending manipulator or wing member on the housing.

### BRIEF SUMMARY OF EMBODIMENTS

It is an object of the present disclosure to provide a treatment tool which effectively prevents an end effector and an elongated member such as sheath or shaft from being rotated by force acting on the end effector.

Accordingly, one aspect of the disclosed technology is directed to a treatment tool comprises a housing and an elongated member configured to be coupled to the housing. The elongated member such as sheath or shaft is capable of rotating around an axis of rotation with respect to the housing. An end effector is disposed on a distal-end portion of the elongated member such as sheath or shaft. A rotary member is configured to be coupled to the elongated member and the housing. The rotary member includes at least one ridge and one cavity. The ridge projects toward an outer circumferential side and extends from a proximal-end side toward a distal-end side on the rotary member. The cavity is formed on the distal-end side from the ridge on the rotary member. The rotary member is capable of rotating in unison with the elongated member such as sheath or shaft and the end effector around the axis of rotation.

Another aspect of the disclosed technology is directed to a treatment tool comprises a housing and an elongated member configured to be coupled to the housing and is capable of rotating around an axis of rotation with respect to the housing. An end effector is disposed on a distal-end portion of the elongated member. A rotary member is configured to be coupled to the elongated member and the housing and is capable of rotating in unison with the elongated member such as sheath or shaft and the end effector around the axis of the rotation. The rotary member has at least one ridge and at least one cavity. The at least one cavity is disposed on a distal-end side of the ridge.

A further aspect of the disclosed technology is directed to a treatment tool comprises a housing having at least one cavity. An elongated member such as sheath or shaft is configured to be coupled to the housing and is capable of rotating around an axis of rotation with respect to the housing. An end effector is disposed on a distal-end portion of the elongated member. A rotary member is configured to be coupled to the elongated member and the housing. The rotary member includes at least one ridge that projects toward an outer circumferential side and extends from a proximal-end side toward a distal-end side on the rotary member and is formed on the proximal-end side from the cavity. The rotary member is capable of rotating in unison with the elongated member and the end effector around the axis of rotation.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic view of a treatment tool according to a first embodiment, as viewed from one side of a housing thereof in its widthwise directions.

FIG. 2 is a schematic view of the treatment tool according to the first embodiment, as viewed from a side opposite the side where a grip is positioned, across a predetermined axis of rotation.

FIG. 3 is a schematic view of a rotary member according to the first embodiment, as viewed from a proximal-end side thereof.

FIG. 4 is a cross-sectional view of the rotary member according to the first embodiment, taken along a cross section substantially perpendicular to the predetermined axis of rotation.

FIG. 5 is a cross-sectional view taken alone line V-V of FIG. 3.

FIG. 6 is a cross-sectional view taken alone line VI-VI of FIG. 3.

FIG. 7 is a schematic view of the housing according to the first embodiment that is held by a hand, as viewed from one side of the housing in its widthwise directions.

FIG. 8 is a cross-sectional view of the rotary member with an index finger abutting against the rotary member, taken along a cross section substantially parallel to the predetermined axis of rotation.

FIG. 9 is a perspective view schematically illustrating a treatment tool according to a first modification.

FIG. 10 is a perspective view schematically illustrating the manner in which a housing of the treatment tool according to the first modification is held by a hand.

FIG. 11 is a schematic view illustrating the structure of a distal-end portion of a treatment tool according to a second modification.

FIG. 12 is a schematic view illustrating the structure of a distal-end portion of a treatment tool according to a third modification.

FIG. 13 is a schematic view illustrating the structure of a distal-end portion of a treatment tool according to a fourth modification.

### DETAILED DESCRIPTION OF THE EMBODIMENTS

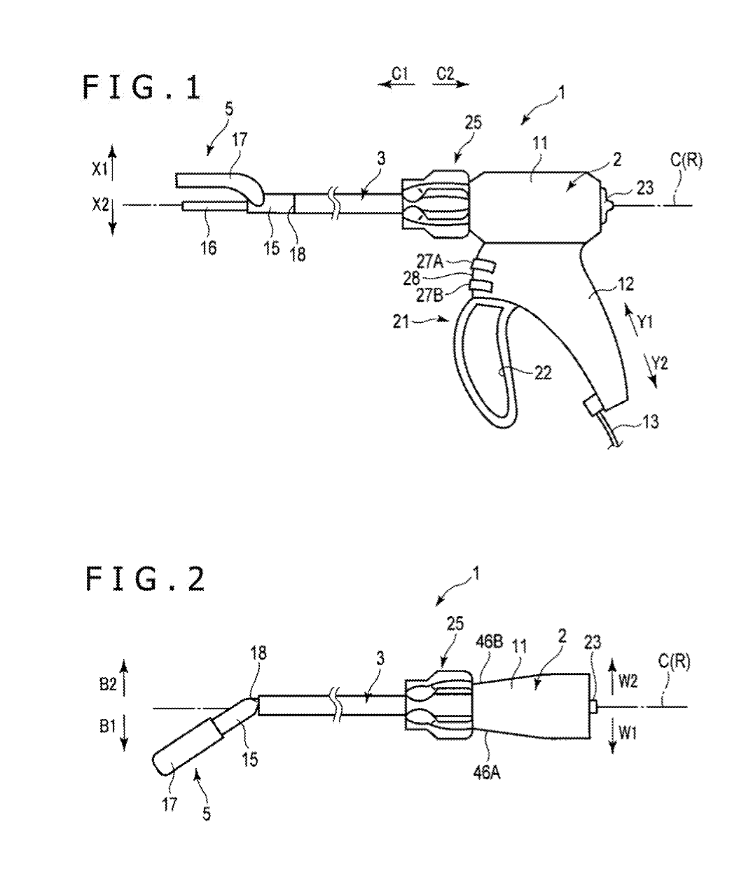

A first embodiment of the present disclosure will be described with reference to FIGS. 1 through 9. FIGS. 1 and 2 are views illustrating a treatment tool or gripping treatment tool 1 according to the present embodiment. As illustrated in FIGS. 1 and 2, the treatment tool 1 has a longitudinal axis C. Of the treatment tool 1, one side in a direction along the longitudinal axis C will be referred to as a distal-end side or arrow C1 side, and a side opposite the distal-end side will be referred to as a proximal-end side or arrow C2 side.

The treatment tool 1 includes a housing 2 that can be held, an elongated member such as a shaft or sheath 3 coupled to a distal-end side of the housing 2, and an end effector 5 disposed on a distal-end portion of the shaft 3. The shaft 3 extends along the longitudinal axis from a proximal-end side to a distal-end side. The elongated member such as the shaft 3 has a central axis that is substantially coaxial with the longitudinal axis C. The shaft 3 has a side extending toward the housing 2 as the proximal-end side and a side extending toward the end effector 5 as the distal-end side. The elongated member such as the shaft 3 is rotatable about its central axis with respect to the housing 2. In other words, the central axis of the shaft 3 is used as an axis of rotation R about which the shaft 3 is rotatable about the housing 2.

The housing 2 includes a housing body 11 extending along the longitudinal axis C, i.e., the axis of rotation R of the shaft 3, and a grip or fixed handle 12 extending from the housing body 11 in directions transverse to the axis of rotation R, i.e., the directions indicated by the arrows Y1, Y2. The grip 12 is disposed in a region spaced from the axis of rotation R, i.e., the longitudinal axis C. A cable 13 has an end connected to the grip 12. The other end of the cable 13 is connected to an energy controller, not depicted. The directions that are transverse to the longitudinal axis C, i.e., the axis of rotation R, or substantially perpendicular thereto and that are also transverse to the direction in which the grip 12 extends, or substantially perpendicular thereto, will also be referred to as widthwise directions of the housing 2, i.e., the directions indicated by the arrows W1, W2. FIG. 1 is a view of the treatment tool 1 as viewed from one side in a widthwise direction of the housing 2, i.e., an arrow W1 side. FIG. 2 is a view of the treatment tool 1 as viewed from a side opposite the side where the grip 12 is positioned, across the axis of rotation R, i.e., the longitudinal axis C.

The end effector 5 is rotatable in unison with the elongated member such as the shaft 3 about the axis of rotation R with respect to the housing 2. The end effector 5 is also bendable with respect to the shaft 3, i.e., the axis of rotation R. When the end effector 5 rotates, the angular position of the end effector 5 about the axis of rotation R is changed. The directions in which the end effector 5 is bendable, i.e., the directions indicated by the arrows B1, B2, are transverse to the axis of rotation R or substantially perpendicular thereto. The end effector 5 includes a relay member 15, a first gripping member 16, and a second gripping member 17. The relay member 15 is attached to the distal end of the elongated member such as the shaft 3 such that the relay member 15 is bendable with respect to the shaft 3. In other words, a bendable joint 18 is formed between the shaft 3 and the relay member 15. On the end effector 5, the space between the gripping members 16, 17 can selectively be opened and closed. The directions in which the gripping members 16, 17 are opened and closed, i.e., the directions indicated by the arrows X1, X2, are transverse to the axis of rotation R and are also transverse to the directions in which the end effector 5 is bendable.

According to an embodiment, one of the gripping members 16, 17 is integral with or fixed to the relay member 15. The other of the gripping members 16, 17 is angularly movably attached to the relay member 15. According to another embodiment, both the gripping members 16, 17 are angularly movably attached to the relay member 15. According to still another embodiment, a rod, not depicted, extends from within the relay member 15 toward the distal-end side, and a portion of the rod that projects from the relay member 15 toward the distal-end side is used as one of the gripping members 16, 17. The other of the gripping members 16, 17 is angularly movably attached to the relay member 15.

A handle or movable handle 21 is angularly movably mounted on the housing 2. When the handle 21 is angularly moved with respect to the housing 2, the handle 21 is opened or closed with respect to the grip 12. In other words, the handle 21 is openable and closable with respect to the grip 12. The handle 21 has a force applying portion 22 to which a manipulating force for opening or closing the handle 21 with respect to the grip 12 is applicable. According to the present embodiment, since the treatment tool 1 is pistol-shaped, the force applying portion 22 is positioned on one side of the axis of rotation R, i.e., the longitudinal axis C, where the grip 12 is positioned, and on the distal-end side with respect to the grip 12. When the handle 21 is opened and closed with respect to the grip 12, the handle 21 is moved in directions substantially parallel to the longitudinal axis C. When a manipulating force is applied to the force applying portion 22, opening or closing the handle 21 with respect to the grip 12, a movable member, not depicted, extending in the shaft 3 moves along the longitudinal axis C, i.e., the axis of rotation R, with respect to the shaft 3 and the housing 2. At least one of the gripping members 16, 17 is now angularly moved with respect to the relay member 15, opening or closing the space between the gripping members 16, 17.

A bending dial 23 as a bending manipulation input portion is mounted on the housing 2. When a manipulation input is applied through the bending dial or bending manipulation input portion 23 by angularly moving the bending dial 23, for example, a bending wire, not depicted, extending in the elongated member such as the shaft 3 moves along the longitudinal axis C, i.e., the axis of rotation R, with respect to the shaft 3 and the housing 2. The end effector 5 is now bent with respect to the shaft 3. The bending dial 23 may be rotatable in unison with the shaft 3 and the end effector 5 about the axis or rotation R with respect to the housing 2, or may not be rotatable in unison with the shaft 3 and the end effector 5 about the axis or rotation R. According to the present embodiment, the bending dial 23 is mounted on a proximal-end face of the housing body 11. However, the position of the bending dial 23 is not limited to the illustrated position. A bending manipulation input portion such as a bending dial 23 or the like may be mounted on an outer surface of the housing body 11 which faces a side opposite to the side of the axis of rotation R, i.e., the longitudinal axis C, where the grip 12 is positioned.

A rotary member or rotary knob 25 is mounted on the distal-end side of the housing body 11. The shaft 3 is mounted on the housing 2 while being inserted from the distal-end side into the rotary member 25 and the housing body 11. The rotary member 25 is attached to the shaft 3. The rotary member 25 is rotatable in unison with the shaft 3 and the end effector 5 about the axis of rotation R with respect to the housing 2. According to the present embodiment, a manipulating force tending to rotate the shaft 3 and the end effector 5 about the axis of rotation R is applied to the rotary member 25 that is used as a rotational manipulation input member.

Manipulating buttons 27A, 27B are mounted on the housing 2. Each of the manipulating buttons 27A, 27B enters a manipulation input when it is pressed. The housing 2 has, on an outer surface thereof, a mount surface 28 that faces the distal-end side at a position between the force applying portion 22 of the handle 21 and the rotary member 25. The manipulating buttons 27A, 27B are disposed on the mount surface 28. Consequently, each of the manipulating buttons 27A, 27B is spaced from the axis of rotation R, i.e., the longitudinal axis C, by a smaller distance than the force applying portion 22 of the handle 21. In other words, each of the manipulating buttons 27A, 27B is closer to the axis of rotation R. One of the manipulating buttons 27A, 27B may not be disposed on the mount surface 28, but may be disposed on an area of the outer surface of the housing 2 which faces one side in the widthwise directions. In this case, too, each of the manipulating buttons 27A, 27B is spaced from the axis of rotation R by a smaller distance than the force applying portion 22 of the handle 21.

When each of the manipulating buttons 27A, 27B enters a manipulation input, the treatment tool 1 operates in a predetermined mode of operation. At this time, as is the case with the known treatment tool, for example, the treatment tool 1 applies either a high-frequency current, an ultrasonic vibration, or heater-generated heat as treatment energy to a treatment target gripped between the gripping members 16, 17. According to an embodiment, when treatment tool 1 operates in a predetermined mode of operation based on a manipulation input from either one of the manipulating buttons 27A, 27B, an electric motor may be energized to stick a staple into the treatment target gripped between the gripping members 16, 17.

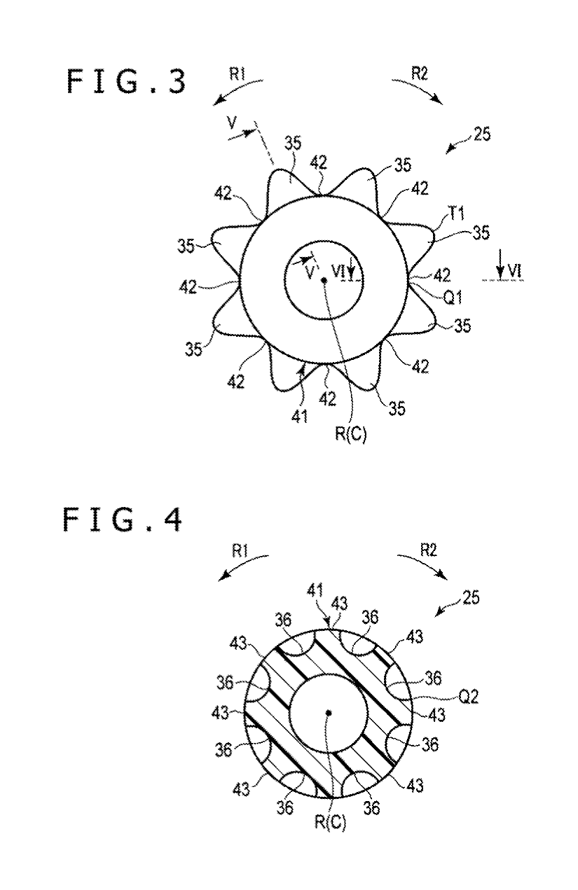

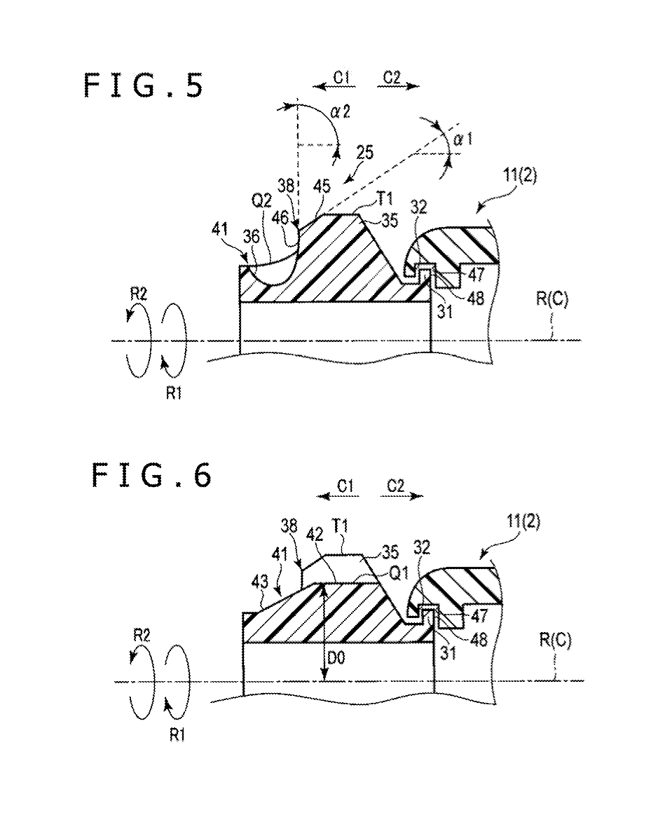

FIGS. 3 through 6 are views illustrating the structure of the rotary member 25. FIG. 3 is a view of the rotary member or rotary knob 25, as viewed from the proximal-end side thereof or the arrow C2 side. FIG. 4 is a cross-sectional view of the rotary member 25, taken along a cross section substantially perpendicular or transverse to the axis of rotation R. FIG. 5 is a cross-sectional view taken alone line V-V of FIG. 3, and FIG. 6 is a cross-sectional view taken alone line VI-VI of FIG. 3. As depicted in FIGS. 5 and 6, the rotary member 25 has an engaging ledge 31 projecting from the proximal-end of an outer circumferential surface thereof toward an outer circumferential side thereof. The engaging ledge 31 extends fully circumferentially around the axis of rotation R. The housing body 11 has an engaging recess 32 defined in a distal-end portion of an inner circumferential surface thereof and being recess toward an outer circumferential side thereof. The engaging recess 32 extends fully circumferentially around the axis of rotation R. The rotary member 25 has a distal-end portion inserted in the housing body 11, with the engaging ledge 31 engaging in the engaging recess 32. The engaging ledge 31 is movable in the engaging recess 32 around the axis of rotation R. Since the engaging ledge 31 engages in the engaging recess 32, the rotary member 25 is mounted on the housing 2 so as to be rotatable about the axis of rotation R.

As illustrated in FIGS. 3 through 6, the rotary member 25 has a plurality of, i.e., eight in the present embodiment, ridges 35 disposed on the outer circumferential surface thereof and a plurality of, i.e., eight in the present embodiment, cavities 36 defined in the outer circumferential surface thereof. Each of the ridges 35 projects toward the outer circumferential side of an arbitrary reference plane. Each of the cavities 36 is concave toward the inner circumferential side of the arbitrary reference plane. The ridges 35 and the cavities 36 are positioned on the distal-end side of the engaging ledge 31 and are exposed outwardly. The ridges 35 are spaced apart from each other around the axis of rotation R, i.e., the longitudinal axis C. The cavities 36 are spaced apart from each other around the axis of rotation R. Each of the ridges 35 extends from the proximal-end side toward the distal-end side. According to the present embodiment, the number of the ridges 35 is the same as the number of the cavities 36. Each of the cavities 36 is contiguous to the distal-end side of a corresponding one of the ridges 35. Therefore, one cavity 36 is contiguous to the distal-end side of each of the ridges 35. The ridges 35 should preferably be disposed symmetrically around the axis of rotation R, i.e., the longitudinal axis C. The cavities 36 should preferably be disposed symmetrically around the axis of rotation R. The ridges 35 are disposed at substantially equal spaced intervals around the axis of rotation R. The cavities 36 are also disposed at substantially equal spaced intervals around the axis of rotation R. Each of the cavities 36 is shaped to fit over the pad of an index finger. FIG. 4 illustrates a cross section substantially perpendicular to the axis of rotation R, which extends through the cavities 36. FIG. 5 illustrates a cross section substantially parallel to the longitudinal axis C, which extends through a ridge, i.e., one of the ridges 35, and a cavity, i.e., one of the cavities 36, that is contiguous to the distal-end side of that ridge, i.e., one of the ridges 35.

Each of the ridges 35 has a protrusive end T1 and a base Q1. On each of the ridges 35, the distance from the axis of rotation R is the largest at the protrusive end T1, and the distance from the axis of rotation R is the smallest at the base Q1. The rotary member 25 has on its outer circumferential surface a defining face 41 that defines the base Q1 of each of the ridges 35. The defining face 41 is exposed outwardly. According to the present embodiment, the defining face 41 is of an arcuate shape about the axis of rotation R at its center, as viewed in the directions along the axis of rotation R, i.e., toward the distal-end side and the proximal-end side, or in a cross section perpendicular to the axis of rotation R. The defining face 41 extends at least in a range from the proximal end of the ridge 35 to the distal end of the cavity 36 in the directions along the axis of rotation R, i.e., the longitudinal axis C. The defining face 41 extends smoothly without radial steps from the proximal end to the distal end. According to an embodiment, the defining face 41 has a radial distance D0 from the axis of rotation R that is substantially uniform from the proximal end to the distal end of the defining face 41. According to another embodiment, the defining face 41 includes a distance varying portion in which the radial distance D0 from the axis of rotation R is gradually smaller from the proximal end to the distal end.

In any of the embodiments, each of the ridges 35 projects from the defining face 41 toward the outer circumferential side within the range in which the ridge 35 extends in the directions along the axis of rotation R. Therefore, each of the ridges 35 and another ridge, i.e., a corresponding one of the ridges 35, adjacent thereto on one side, i.e., the arrow R1 side, around the axis of rotation R define therebetween a valley, a corresponding one of valleys 42, that is recess toward the inner circumferential side. In other words, in the cross section substantially perpendicular to the axis of rotation R, which extends through the ridges 35, the ridges 35 and the valleys 42 are alternately disposed around the axis of rotation R, i.e., the longitudinal axis C. The valleys 42 are defined by the defining faces 41.

Each of the cavities 36 is concave from the defining face 41 toward the inner circumferential side within the range in which the cavity 36 extends in the directions along the axis of rotation R. Each of the cavities 36 is open at the defining face 41 toward the outer circumferential side. Each of the cavities 36 has an opening edge Q2 defined by the defining face 41. Therefore, each of the cavities 36 and another cavity, i.e., a corresponding one of the cavities 36, adjacent thereto on one side, i.e., the arrow R1 side, around the axis of rotation R define therebetween a projection, a corresponding one of projections 43, that projects toward the outer circumferential side. In other words, in the cross section substantially perpendicular to the axis of rotation R, which extends through the cavities 36, the cavities 36 and the projections 43 are alternately disposed around the axis of rotation R, i.e., the longitudinal axis C. The projections 43 are defined by the defining faces 41.

As described hereinbefore, each of the cavities 36 is contiguous to the distal-end side of a corresponding one of the ridges 35. Therefore, each of the projections 43 is contiguous to the distal-end side of a corresponding one of the valleys 42. Each of the projections 43 projects toward the outer circumferential side with respect to the cavities 36, and is positioned on the inner circumferential side with respect to the ridges 35. FIG. 6 illustrates a cross section substantially parallel to the longitudinal axis C, which extends through a valley, i.e., one of the valleys 42, and a projection, i.e., one of the projections 43, that is contiguous to the distal-end side of that valley, i.e., one of the valleys 42. The defining face 41 may not necessarily be of an arcuate curved surface about the axis of rotation R at its center, as viewed in the directions along the axis of rotation R. According to an embodiment, the defining face 41 may be of a flat shape. Even in such a case, the defining face 41 defines the base Q1 of the ridge 35 and the opening edge Q2 of the cavity 36.

According to the present embodiment, as described hereinbefore, each of the ridges 35 projects toward the outer circumferential side from the defining face 41. Each of the cavities 36 is contiguous to the distal-end side of a corresponding one of the ridges 35, and is concave toward the inner circumferential side from the defining face 41. Consequently, a radial step 38 or a corresponding one of steps 38 is formed between each of the ridges 35 and a cavity 36 or a corresponding one of the cavities 36 that is contiguous to the distal-end side of the ridge 35. Each of the steps 38 is larger than a radial step between each of the ridges 35 and a corresponding valley 42 or a corresponding one of the valleys 42.

Each of the steps 38 has a first slanted surface 45 or a corresponding one of first slanted surfaces 45 and a second slanted surface 46 or a corresponding one of second slanted surfaces 46. Each of the first slanted surfaces 45 extends from the distal-end portion of a corresponding ridge 35 or a corresponding one of the ridges 35 toward the distal-end side. Each of the second slanted surfaces 46 is contiguous to the distal-end side of a corresponding first slanted surface 45 or a corresponding one of the first slanted surfaces 45, and extends toward the distal-end side up to a corresponding cavity 36 or a corresponding one of the cavities 36. Each of the first slanted surfaces 45 and the second slanted surfaces 46 is slanted toward the inner circumferential side gradually from the proximal-end side toward the distal-end side. In other words, the distance of each of the first slanted surfaces 45 and the second slanted surfaces 46 from the axis of rotation R is reduced gradually toward the distal-end side. Each of the first slanted surfaces 45 is inclined to the axis of rotation R at a first tilt angle or acute angle α1. Each of the second slanted surfaces 46 is inclined to the axis of rotation R at a second tilt angle or acute angle α2 that is larger than the first tilt angle α1. Therefore, the gradient of each of the second slanted surfaces 46 is larger than the gradient of each of the first slanted surfaces 45.

The housing 2 has a first confronting face 47 in the engaging recess 32 that faces toward the distal-end side. The first confronting face 47 confronts the engaging ledge 31 of the rotary member 25. The engaging ledge 31 of the rotary member 25 has a second confronting face 48 that confronts the first confronting face 47. The second confronting face 48 faces toward the proximal-end side. When a force is applied to the rotary member 25 toward the proximal-end side, the rotary member 25 slightly moves toward the proximal-end side with respect to the housing 2. The second confronting face 48 thus abuts against the first confronting face 47.

According to an embodiment, the first confronting face 47 of the housing 2 may face one side, e.g., the arrow W1 side, in a widthwise direction, and the second confronting face 48 of the rotary member 25 may face the first confronting face 47. In this case, when a force is applied to the rotary member 25 in one of the widthwise directions, i.e., the directions of the arrows W1, W2, of the housing 2, the rotary member 25 slightly moves with respect to the housing 2 in the widthwise direction of the housing 2. The second confronting face 48 thus abuts against the first confronting face 47.

Next, operation and advantages of the treatment tool 1 according to the present embodiment will be described. For treating a treatment target such as a living tissue or the like using the treatment tool 1, the operator holds the housing 2 with one hand thereof, i.e., the left hand or the right hand thereof, and inserts the end effector 5 into a body cavity such as an abdominal cavity or the like. The operator then rotates the rotary member 25 to rotate the shaft 3 and the end effector 5 about the axis of rotation R, and operates the bending dial 23 to bend the end effector 5 with respect to the shaft 3, thereby adjusting the position and posture of the end effector 5 in the body cavity. After having adjusted the position and posture of the end effector 5 in a manner to place the treatment target between the gripping members 16, 17, the operator closes the handle 21 with respect to the grip 12, closing the space between the gripping members 16, 17. The treatment target is now gripped between the gripping members 16, 17. With the treatment target being gripped, the operator presses the manipulating button 27A or 27B to enter a manipulation input, operating the treatment tool 1 in a predetermined mode of operation. Treatment energy such as a high-frequency current is applied to or a staple is stuck into the treatment target gripped as described hereinbefore.

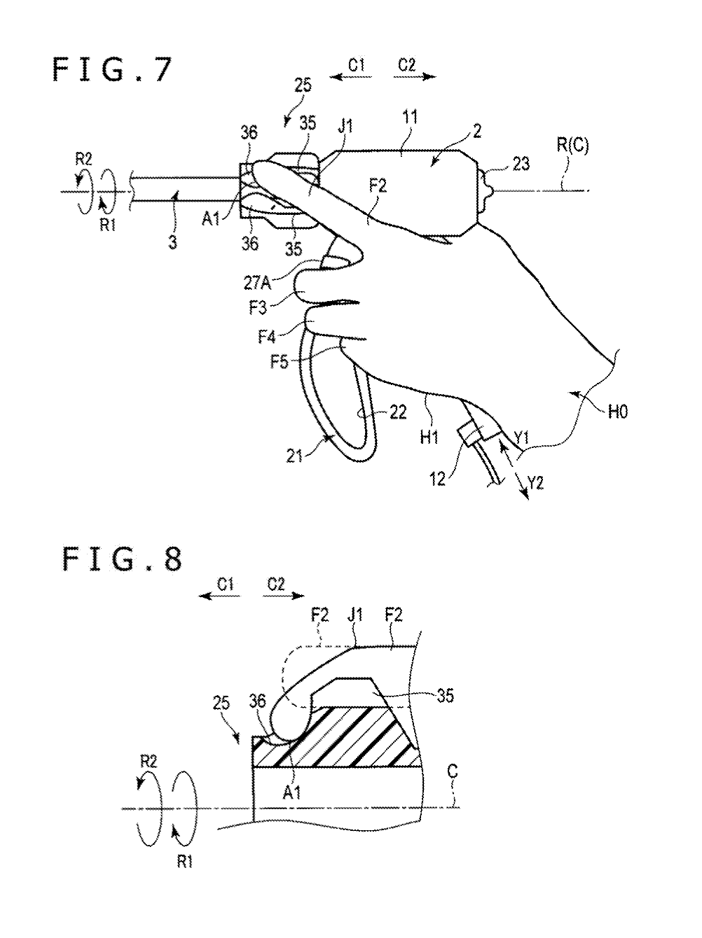

FIG. 7 is a view of the housing 2 of the treatment tool 1 that is held with one hand H0, i.e., the left hand in FIG. 7. FIG. 7 is viewed from one side, i.e., the arrow W1 side, in the widthwise directions of the housing 2. FIG. 7 illustrates the manner in which the index finger F2 is held against a cavity 36, i.e., one of the cavities 36. FIG. 8 is a cross-sectional view of the rotary member or rotary knob 25 with the index finger F2 abutting against the rotary member 25, taken along a cross section substantially parallel to the axis of rotation R or the longitudinal axis C. In FIG. 8, the index finger F2 held against a ridge 35, i.e., one of the ridges 35, of the rotary member 25 is indicated by the broken line, and the index finger F2 held against a cavity 36, i.e., one of the cavities 36, in the rotary member 25 is indicated by the solid line.

As depicted in FIG. 7, when the housing 2 is held by one hand H0, e.g., the left hand, the palm H1 and the thumb F1 are held against the grip 12 from the proximal-end side. The third finger F4 and the little finger F5 are placed on the force applying portion 22 of the handle 21, and the third finger F4 and/or the little finger F5 applies a manipulating force for opening or closing the handle 21 with respect to the grip 12 to the force applying portion 22. For bending the end effector 5 with respect to the shaft 3, the thumb F1 operates the bending dial or bending manipulation input portion 23. Each of the manipulating buttons 27A, 27B is pressed by the middle finger F3, which enters a manipulation input from each of the manipulating buttons 27A, 27B. If the manipulating button 27A or 27B is disposed on a portion of the outer surface of the housing 2 which faces one side in the widthwise directions, then the thumb F1 enters a manipulation input from the manipulating button 27A or 27B.

For adjusting the angular position of the end effector 5 about the axis of rotation R, the pad A1 of the index finger F2, i.e., a portion of the index finger F2 which extends from the distal interphalangeal (DIP) joint J1 toward the fingertip, is held against either one of the ridges 35 of the rotary member 25. Then, the pad A1 of the index finger F2 applies a manipulating force for rotating the rotary member 25 about the axis of rotation R or the longitudinal axis C, to the rotary member 25. The shaft 3 and the end effector 5 are now rotated in unison with the rotary member 25 about the axis of rotation R with respect to the housing 2. At this time, the index finger F2 extends substantially straight without bending at the distal interphalangeal (DIP) joint or first joint J1.

According to the present embodiment, each of the first slanted surfaces 45 extends from the distal end of a corresponding ridge 35, i.e., a corresponding one of the ridges 35, toward the distal-end side. The first tilt angle or acute angle α1 of each of the first slanted surfaces 45 with respect to the axis of rotation R is small. Therefore, when the rotary member 25 is to be rotated about the axis of rotation R, it is easy for the pad A1 of the index finger F2 to move from the ridge 35 which the pad A1 of the index finger F2 has been abutting against to a ridge 35 or a corresponding one of the ridges 35 that is adjacent about the axis of rotation R. Consequently, the ease of manipulation with which to rotate the rotary member 25 is increased.

In a treatment using the treatment tool 1, a force may act on the end effector 5 while the end effector 5 is being bent with respect to the shaft 3 or the axis of rotation R. Since the force acts on the end effector 5 at a position spaced from the central axis of the shaft 3 or the axis of rotation R, an angular moment may be produced around the axis of rotation R, i.e., the central axis of the shaft 3, by the force acting on the end effector 5. When an angular moment is produced around the axis of rotation R by the force acting on the end effector 5, the operator bends the pad A1 of the index finger F2 out of abutment against a ridge 35 or one of the ridges 35 toward the inner circumferential side of the rotary member 25 at the distal interphalangeal joint J1. Then, as illustrated in FIGS. 7 and 8, the operator brings the fingertip portion, i.e., the distal-end portion, of the pad A1 of the index finger F2 into one of the cavities 36.

Each of the ridges 35 projects toward the outer circumferential side from the defining face 41. Each of the cavities 36 is contiguous to the distal-end side of a corresponding one of the ridges 35 and is concave toward the inner circumferential side from the defining face 41. Therefore, while the index finger F2 is being placed in a cavity 36 or one of the cavities 36, the pad A1 of the index finger F2 is bent at the distal interphalangeal joint J1 toward the inner circumferential side of the rotary member 25 with respect to the portion of the index finger F2 that is closer to its base than the distal interphalangeal joint J1. As the ridges 35 and the cavities 36 are disposed as described hereinbefore, when the operator bends the pad A1 of the index finger F2 out of abutment against a ridge 35 or one of the ridges 35 toward the inner circumferential side at the distal interphalangeal joint J1, the pad A1 of the index finger F2 is placed in a cavity 36 or one of the cavities 36. Therefore, at least one of the cavities 36 is positioned in a range in which the index finger F2 can be placed therein while the housing 2 is being held by the hand H0.

With a pistol-shaped treatment tool such as the treatment tool 1, the rotary member 25 is coupled to the distal-end side of the housing body 11, and the grip 12 is disposed in a region spaced from the axis of rotation R or the longitudinal axis C. The force applying portion 22 is positioned on the side of the axis of rotation R or the longitudinal axis C where the grip 12 is positioned, and is positioned on the distal-end side with respect to the grip 12. Consequently, while the housing 2 is being held by one hand H0 and the pad A1 of the index finger F2 is being placed in a cavity 36 or one of the cavities 36, the index finger F2 extends obliquely to the axis of rotation R or the longitudinal axis C, as viewed from one side or the arrow W1 side or the arrow W2 side in a direction that is traverse to the axis of rotation R and also to the directions along which the grip 2 extends, i.e., the directions indicated by the arrows Y1, Y2.

As described hereinbefore, when an angular moment is produced around the axis of rotation R by the force acting on the end effector 5, the pad A1 of the index finger F2 that extends obliquely to the axis of rotation R, as viewed from one side in a widthwise direction of the housing 2, is placed in a cavity 36 or one of the cavities 36. The pad A1 of the index finger F2 is thus bent toward the inner circumferential side of the rotary member 25 at the distal interphalangeal (DIP) joint or first joint J1. The bent index finger F2 makes it easier to apply, to the rotary member 25, a restraining force tending to restrain the rotation of the shaft 3 and the end effector 5 due to the angular moment, than the index finger F2 that is not bent, i.e., that extends substantially straight. Consequently, when the pad A1 of the index finger F2 is bent toward the inner circumferential side of the rotary member 25 at the distal interphalangeal (DIP) joint J1, the restraining force that acts on the rotary member 25 from the index finger F2 in a direction opposite the angular moment becomes large. A torque tending to cancel the rotation of the shaft 3 and the end effector 5 due to the angular moment, i.e., the product of the restraining force and the distance from the axis of rotation R to the position where the restraining force is applied, is thus large. Therefore, even when the angular moment is produced around the axis of rotation R by the force acting on the end effector 5, the restraining force applied from the index finger F2 to the rotary member 25 effectively prevents the shaft 3 and the end effector 5 from being rotated about the axis of rotation R. The performance of a treatment that is performed while the treatment target is gripped between the gripping members 16, 17 is thus adequately maintained.

When an angular moment is produced toward one side, e.g., the arrow R1 side, around the axis of rotation R by a force acting on the end effector 5, the fingertip portion of the pad A1 of the index finger F2 placed in a cavity 36 or one of the cavities 36 applies a restraining force tending to restrain the rotation of the shaft 3 and the end effector 5 due to the angular moment. While the housing 2 is being held by one hand H0 and the fingertip portion of the pad A1 of the index finger F2 is being placed in the one of the cavities 36, the pad A1 extends toward the proximal-end side over a projection 43 or a corresponding one of the projections 43 that is adjacent to the cavity 36 or the one of the cavities 36 in which the fingertip portion of the pad A1 is placed, on one side or the arrow R1 side around the axis of rotation R. The distal interphalangeal joint J1 of the index finger F2 or a nearby portion of the index finger F2, e.g., a base portion of the pad A1, is held against one of the ridges 35. A cavity 36 or a corresponding one of the cavities 36 that is contiguous to the distal-end side of the ridge 35 or the corresponding one of the ridges 35 against which the distal interphalangeal joint J1 of the index finger F2 or the nearby portion of the index finger F2 is held is disposed adjacent to the cavity 36 or the one of the cavities 36 in which the fingertip portion of the pad A1 is placed, on one side around the axis of rotation R. In other words, the cavity 36 or the one of the cavities 36 in which the fingertip portion of the pad A1 is placed and the ridge 35 or the corresponding one of the ridges 35 against which the distal interphalangeal joint J1 of the index finger F2 or the nearby portion of the index finger F2 is held are disposed adjacent to each other around the axis of rotation R.

When an angular moment is produced toward the other side, e.g., the arrow R2 side, around the axis of rotation R by a force acting on the end effector, as described hereinbefore, the distal interphalangeal joint or first joint J1 or a nearby portion thereof that is held against a ridge 35 or a corresponding one of the ridges 35 applies a restraining force tending to restrain the rotation of the shaft 3 and the end effector 5 due to the angular moment.

Each of the cavities 36 is shaped to fit over the pad A1 of the index finger F2. Therefore, when the fingertip portion of the pad A1 of the index finger F2 is fitted in a cavity 36 or one of the cavities 36, it is easy for the pad A1 to remain bent at the distal interphalangeal joint J1. According to the present embodiment, furthermore, each of the second slanted surfaces 46 is contiguous to a corresponding first slanted surface 45 or one of the first slanted surfaces 45, and extends toward the distal-end side up to a corresponding cavity 36 or a corresponding one of the cavities 36. The second tilt angle or acute angle α2 of each of the second slanted surfaces 46 with respect to the axis of rotation R is large and close to 90°. Since the second tilt angle α2 is large, the depression of the cavity 36 from the defining face 41 is large, and a radial step 38 or a corresponding one of steps 38 that is formed between each of the ridges 35 and a cavity 36 or a corresponding one of the cavities 36 that is contiguous to the distal-end side of the ridge 35 is also large. As a consequence, it is easy for the fingertip portion of the pad A1 of the index finger F2 to fit in the cavity 36. Therefore, when the pad A1 of the index finger F2 is fitted in a cavity 36 or one of the cavities 36, it is easy for the pad A1 to remain bent at the distal interphalangeal joint J1. As the pad A1 adequately remains bent toward the inner circumferential side at the distal interphalangeal joint J1, it is easy to apply a restraining force to the rotary member 25, and the restraining force can be increased.

With the cavities 36 being disposed symmetrically around the axis of rotation R, even if the end effector 5 is placed in any angular position about the axis of rotation R, at least one cavity 36 or one of the cavities 36 is positioned in a position where the pad A1 of the index finger F2 can be fitted with ease.

According to the present embodiment, while the pad A1 of the index finger F2 is being placed in a cavity 36 or one of the cavities 36, a force directed toward the proximal-end side is applied from the pad A1 to the rotary member 25. At this time, the force is applied to a step 38 or a corresponding one of the steps 38 on the proximal-end side of the cavity 36 or the one of the cavities 36 in which the pad A1 is fitted. As described hereinbefore, while the housing 2 is being held by the hand H0, since the palm H1 and the thumb F1 are held against the grip 12 from the proximal-end side, the palm H1 applies a pressing force to the housing 2 toward the distal-end side. When the force is applied to the rotary member 25 toward the proximal-end side, therefore, the rotary member 25 slightly moves toward the proximal-end side with respect to the housing 2, bringing the second confronting face 48 into abutment against the first confronting face 47. Inasmuch as the second confronting face 48 is brought into abutment against the first confronting face 47, when an angular moment is produced around the axis of rotation R by a force acting on the end effector 5, friction occurs between the first confronting face 47 and the second confronting face 48. As the friction occurs as well as the restraining force applied, the end effector 5 and the shaft 3 are effectively prevented from being rotated about the axis of rotation R by the angular moment.

According to an embodiment, while the pad A1 of the index finger F2 is being placed in a cavity 36 or one of the cavities 36, a force may be applied from the pad A1 to the rotary member 25 in a widthwise direction of the housing 2, thereby bringing the first confronting face 47 and the second confronting face 48 into abutment against each other.

In this case, while the first confronting face 47 and the second confronting face 48 are abutting against each other, if an angular moment is produced around the axis of rotation R by a force acting on the end effector 5, then friction also occurs between the first confronting face 47 and the second confronting face 48.

### Modifications

In the embodiment described hereinbefore, the rotary member 25 has the eight ridges 35 and the eight cavities 36. However, the number of ridges 35 and the number of cavities 36 are not limited to these numerical values. The rotary member may have nine or more ridges 35 and nine or more cavities 36, or seven or less ridges 35 and seven or less cavities 36. Alternatively, the rotary member may have only one ridge 35 and only one cavity 36. In either case, the number of ridges 35 and the number of cavities 36 are the same as each other, and each of the cavities 36 is contiguous to the distal-end side of a corresponding one of the ridges 35.

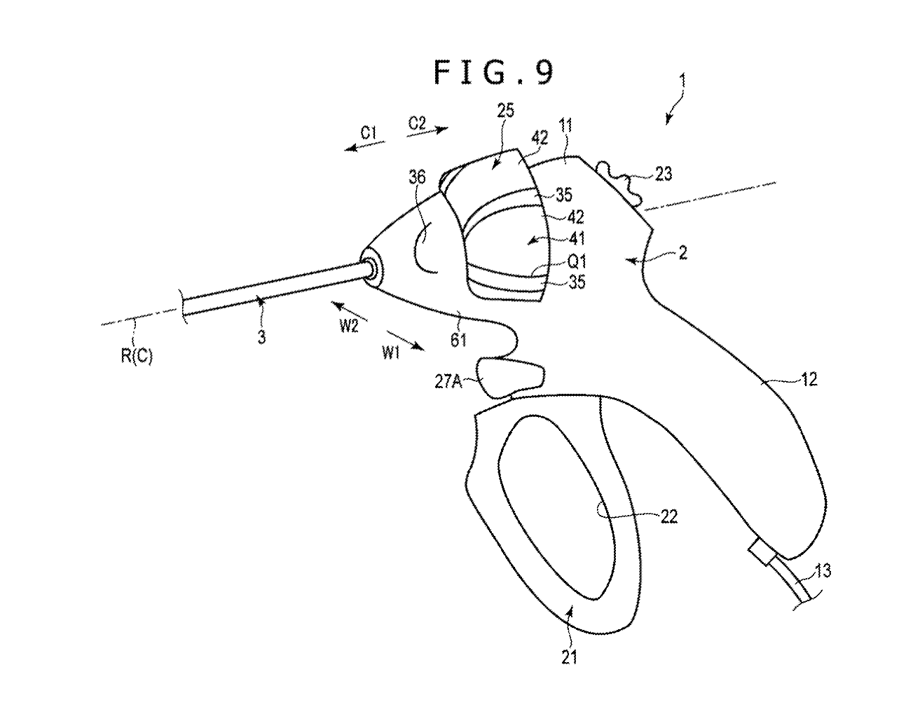

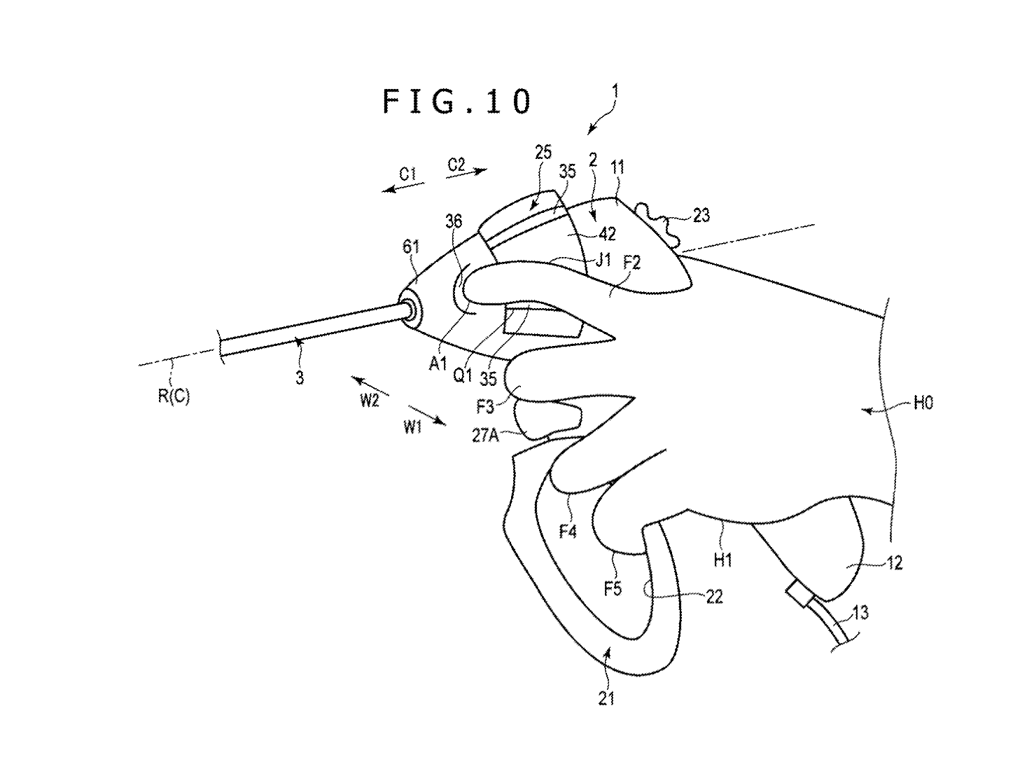

The cavities 36 may not necessarily be defined in the rotary member 25. According to a first modification illustrated in FIG. 9, the housing 2 has cavities 36 (two in the present modification) that are concave toward the inner circumferential side. In the present modification, the rotary member 25 is mounted on the housing body 11, which extends along the axis of rotation R of the rotary member 25 and the shaft 3. According to the present modification, however, the housing body 11 has a distal-end-side outer surface 61 on a distal-end side thereof with respect to the rotary member 25, and the housing body 11 extends over a region toward the distal-end side from the distal end of the rotary member 25. Each of the cavities 36 is concave toward the inner circumferential side in the distal-end-side outer surface 61. According to the present modification, one of the cavities 36 is defined in an area of the distal-end-side outer surface 61 which faces one side, i.e., the arrow W1 side, in the widthwise directions, and the other cavity 36 is defined in an area of the distal-end-side outer surface 61 which faces the other side, i.e., the arrow W2 side, in the widthwise directions. Each of the cavities 36 is shaped to fit over the fingertip portion of the pad A1 of the index finger F2.

According to the present modification, as with the first embodiment, the treatment tool 1 has the grip 12 and the handle 21, and has the mount surface 28 that faces the distal-end side at a position between the force applying portion 22 of the handle 21 and the rotary member 25. The manipulating button 27A is disposed on the mount surface 28.

According to the present modification, the rotary member 25 has the ridges 35 and the defining face 41 that defines the base Q1 of each of the ridges 35. In the cross section substantially perpendicular to the axis of rotation R, which extends through the ridges 35, the ridges 35 and the valleys 42 are alternately disposed around the axis of rotation R, i.e., the longitudinal axis C. According to the present modification, as the cavities 36 are defined in the distal-end-side outer surface 61, each of the cavities 36 is disposed on the housing 2 more closely to the distal-end side than the ridges 35. The cavities 36 should preferably be shifted to a side of the axis of rotation R that is opposite to the side where the grip 12 is positioned.

According to the present modification, each of the ridges 35 projects toward the outer circumferential side with respect to the defining face 41 and the distal-end-side outer surface 61 of the housing 2, and each of the cavities 36 is concave toward the inner circumferential side on the distal-end-side outer surface 61. In the present modification, therefore, while the index finger F2 is being placed in a cavity 36 or one of the cavities 36, the pad A1 of the index finger F2 is bent at the distal interphalangeal joint J1 toward the inner circumferential side of the rotary member 25 with respect to the portion of the index finger F2 that is closer to its base than the distal interphalangeal joint J1. According to the present modification, as with the first embodiment, when the index finger F2 is bent, it is easy to apply, to the rotary member 25, a restraining force tending to restrain the rotation of the shaft 3 and the end effector 5 due to the angular moment. Consequently, according to the present modification, as with the first embodiment, even when the angular moment is produced around the axis of rotation R by the force acting on the end effector 5, the restraining force applied from the index finger F2 to the rotary member 25 effectively prevents the shaft 3 and the end effector 5 from being rotated about the axis of rotation R.

According to the present modification, the distance from the distal end of the rotary member 25, i.e., the distal ends of the ridges 35, to the distal end of each of the cavities 36 is not excessively large. For this reason, when the pad A1 of the index finger F2 is bent out of abutment against a ridge 35 or one of the ridges 35 toward the inner circumferential side at the distal interphalangeal joint J1, the pad A1 of the index finger F2 is placed in a cavity 36 or one of the cavities 36. In other words, at least one of the cavities 36 is positioned in a range in which the index finger F2 can be placed therein while the housing 2 is being held by the hand H0. According to the present modification, moreover, when the pad A1 of the index finger F2 is fitted in one of the cavities 36 and the fingertip portion of the pad A1 is placed in the cavity 36 or the one of the cavities 36, the distal interphalangeal joint or first joint J1 of the index finger F1 or a nearby portion thereof abuts against one of the ridges 35 of the rotary member 25.

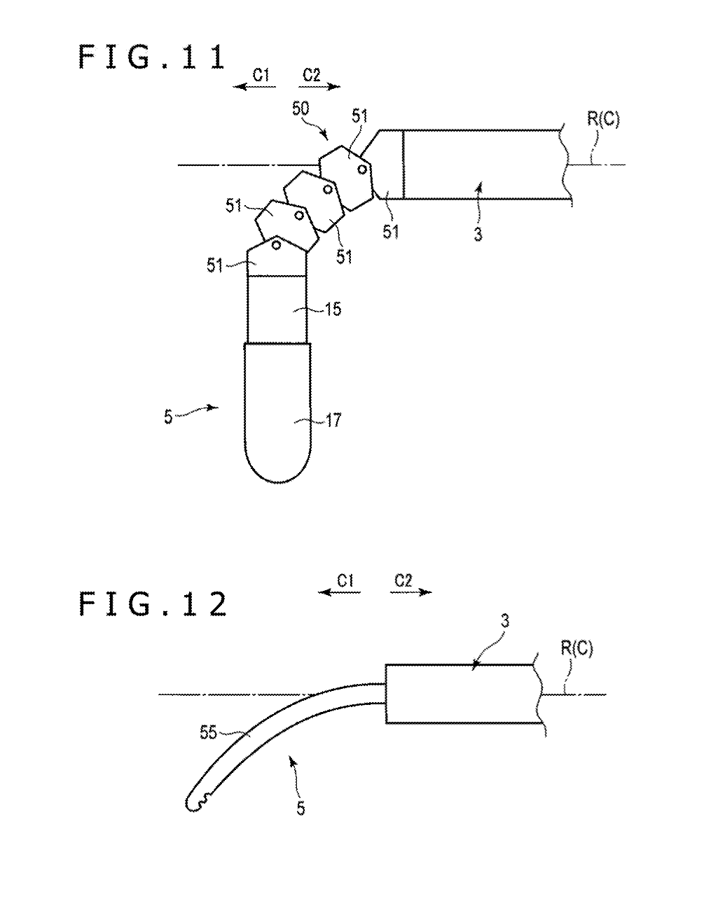

The treatment tool in which an angular moment is produced around the axis R of rotation by a force acting on the end effector 5 is not limited to the treatment tool 1 with the bendable joint 18 according to the embodiment, etc. described hereinbefore. According to a second modification illustrated in FIG. 11, a bendable portion 50 may be disposed in place of the bendable joint 18 on the distal-end side of the shaft 3. The bendable portion 50 is made up of a plurality of arrayed bend elements 51 each angularly movably coupled to an adjacent bend element or elements 51 or a corresponding one or two of the bend elements 51. A bending manipulation input portion is disposed on the housing 2 in place of the bending dial or bending manipulation input portion 23. The bendable portion 50 is actuated by operating the bending manipulation input portion. When the bendable portion 50 is actuated, the end effector 5 that includes the bendable portion 50 is bent with respect to the shaft 3 or the axis of rotation R. When a force acts on the end effector 5 that is being bent with respect to the shaft 3, since the force acts thereon at a position spaced from the axis of rotation R, as described hereinbefore, an angular moment may possibly be produced around the axis of rotation R or the central axis of the shaft 3. According to the present modification, since the treatment tool has the rotary member 25 and the housing 2 that are similar to those of the embodiment, etc. described hereinbefore, even if an angular moment is produced around the axis of rotation R by the force acting on the end effector 5, the index finger F2 whose pad A1 is fitted in a cavity 36 or one of the cavities 36 applies a restraining force to the rotary member 25. Consequently, the end effector 5 and the shaft 3 are effectively prevented from being rotated around the axis of rotation R.

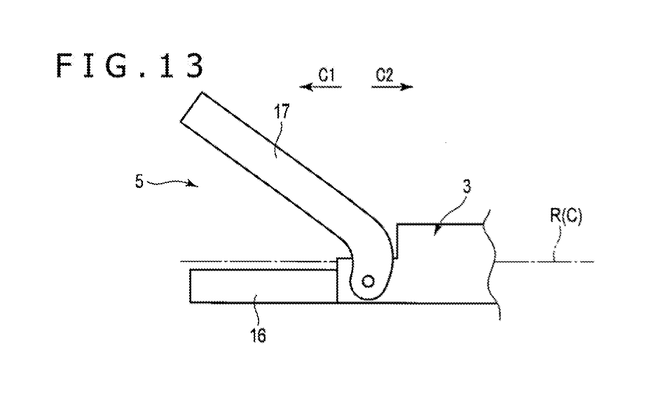

According to a third embodiment illustrated in FIG. 13, an end effector 5 projects toward the distal-end side from the distal end of the shaft 3. The end effector 5 includes a curved extension 55 extending so as to be capable to being curved with respect to the shaft 3 or the axis of rotation R. When a force acts on the end effector 5 at a region on the distal-end side from the curved extension 55, since the force acts thereon at a position spaced from the axis of rotation R, as described hereinbefore, an angular moment may possibly be produced around the axis of rotation R or the central axis of the shaft 3. According to the present modification, since the treatment tool has the rotary member 25 and the housing 2 that are similar to those of the embodiment, etc. described hereinbefore, even if an angular moment is produced around the axis of rotation R by the force acting on the end effector 5, the index finger F2 whose pad A1 is fitted in a cavity 36 or one of the cavities 36 applies a restraining force to the rotary member 25. Consequently, the end effector 5 and the shaft 3 are effectively prevented from being rotated around the axis of rotation R.

According to a fourth modification illustrated in FIG. 13, as with the first embodiment, a pair of gripping members 16, 17 are mounted on an end effector 5. According to the present modification, the treatment tool 1 is devoid of either of the bendable joint 18, the bendable portion 50, and the curved extension 55. The treatment tool 1 with the gripping members 16, 17 may perform a treatment while the space between the gripping members 16, 17 is being open. While the space between the gripping members 16, 17 is being open, if a force acts on a gripping member, e.g., the second gripping member 17, angularly movably mounted on the shaft 3, since the force acts thereon at a position spaced from the axis of rotation R, as described hereinbefore, an angular moment may possibly be produced around the axis of rotation R or the central axis of the shaft 3. According to the present modification, since the treatment tool has the rotary member 25 and the housing 2 that are similar to those of the embodiment, etc. described hereinbefore, even if an angular moment is produced around the axis of rotation R by the force acting on the end effector 5, the index finger F2 whose pad A1 is fitted in a cavity 36 or one of the cavities 36 applies a restraining force to the rotary member 25. Consequently, the end effector 5 and the shaft 3 are effectively prevented from being rotated around the axis of rotation R.

According to the embodiment, etc. described hereinbefore, the treatment tool 1 includes the housing 2 that can be held, the shaft 3 that is rotatable around the axis of rotation R with respect to the housing 2, the end effector 5 disposed on the distal-end portion of the shaft 3, and the rotary member 25 that is rotatable in unison with the shaft 3 and the end effector 5 around the axis of rotation R. The rotary member 25 has the ridges 35 projecting toward the outer circumferential side and extending from the proximal-end side toward the distal-end side. The treatment tool 1 includes the cavities 36 that are concave toward the inner circumferential side, the cavities 36 being contiguous to the distal-end sides of the ridges 35 on the rotary member 25 or being disposed on the distal-end side from the ridges 35 on the housing 2.

In sum, the disclosed technology is directed to a treatment tool comprises a housing and a sheath configured to be coupled to the housing. The sheath is capable of rotating around an axis of rotation with respect to the housing. An end effector is disposed on a distal-end portion of the sheath. A rotary member is configured to be coupled to the sheath and the housing. The rotary member includes at least one ridge and one cavity. The ridge projects toward an outer circumferential side and extends from a proximal-end side toward a distal-end side on the rotary member. The cavity is formed on the distal-end side from the ridge on the rotary member. The rotary member is capable of rotating in unison with the sheath and the end effector around the axis of rotation. The rotary member includes a first slanted surface extends from a distal-end portion of the at least one ridge toward the distal-end side and slanted toward an inner circumferential side of the rotary member gradually from the proximal-end side toward the distal-end side. A second slanted surface is contiguous to a distal-end side of the first slanted surface and extends toward the distal-end side up to the at least one cavity. The second slanted surface is inclined at a larger tilt angle than the first slanted surface toward the inner circumferential side gradually from the proximal-end side toward the distal-end side of the rotary member.

The rotary member includes a face surface that defines a base of the at least one ridge. The at least one cavity is concave toward an inner circumferential side with respect to the face surface. The at least one cavity is shaped so as to correspond to a shape of an index finger. The housing includes a first confronting face that confronts the rotary member. The rotary member has a second confronting face that confronts the first confronting face. The second confronting face abutting against the first confronting face when a force is applied to the rotary member toward the proximal-end side or in a width-wise direction of the housing. The at least one ridge includes a plurality of ridges spaced apart on the rotary member around the axis of rotation. The at least one cavity includes a plurality of cavities spaced apart on the rotary member around the axis of rotation. Each of the plurality of the cavities is contiguous to the distal-end side of a corresponding one of the plurality of the ridges.

The treatment tool further comprises a grip configured to be disposed in a region of the housing spaced apart from the axis of rotation and extending along a direction transverse to the axis of rotation. A movable handle is mounted on the housing so as to be opened and closed with respect to the grip. The movable handle has a force applying portion to which a manipulating force for opening or closing of the movable handle with respect to the grip is applicable. The force applying portion is positioned on a side of the axis of rotation where the grip is positioned and on the distal-end side with respect to the grip. The housing is held by a hand of a user while palm and thumb of the hand are held against the grip from the proximal-end side and the third finger and/or the little finger of the hand applies the manipulating force to the force applying portion. The index finger applies a manipulating force for rotating the rotary member to the ridge. The cavity of the rotary member is positioned in a range in which the index finger of the hand can be placed therein while the hand is holding the housing.

The ridge includes a plurality of ridges spaced apart on the rotary member around the axis of rotation and the cavity includes a plurality of cavities spaced apart on the rotary member around the axis of rotation. Each of the cavities is contiguous to the distal-end side of a corresponding one of the ridges. when the user is holding the housing while a fingertip portion of the index finger is placed in one of the cavities, the distal interphalangeal joint of the index finger or a nearby portion thereof is held against one of the ridges and the cavity that is contiguous to the distal-end side of the ridge against which the distal interphalangeal joint of the index finger or the nearby portion thereof is held being disposed adjacent to the cavity. When the housing is being held by the hand and the index finger of the hand is being placed in the cavity, the index finger extends obliquely to the axis of rotation as viewed from one side in a direction that is traverse to the axis of rotation and also to the direction along which the grip extends. When the housing is being held by the hand and the index finger of the hand is being placed in the cavity, the index finger is bent toward an inner circumferential side of the rotary member at the distal interphalangeal joint of the index finger. The end effector is capable of being bended and/or curved with respect to the sheath.

A treatment tool comprises a housing and a sheath configured to be coupled to the housing and is capable of rotating around an axis of rotation with respect to the housing. An end effector is disposed on a distal-end portion of the sheath. A rotary member is configured to be coupled to the sheath and the housing and is capable of rotating in unison with the sheath and the end effector around the axis of the rotation. The rotary member has at least one ridge and at least one cavity. The at least one cavity is disposed on a distal-end side of the ridge. The ridge has a first surface and the cavity has a second surface contiguous to a distal-end side of the first surface. The second surface is inclined at a larger tilt angle than the first surface.

A treatment tool comprises a housing having at least one cavity. A sheath is configured to be coupled to the housing and is capable of rotating around an axis of rotation with respect to the housing. An end effector is disposed on a distal-end portion of the sheath. A rotary member is configured to be coupled to the sheath and the housing. The rotary member includes at least one ridge that projects toward an outer circumferential side and extends from a proximal-end side toward a distal-end side on the rotary member and is formed on the proximal-end side from the cavity. The rotary member is capable of rotating in unison with the sheath and the end effector around the axis of rotation. The treatment tool further comprises a grip configured to be disposed in a region of the housing spaced apart from the axis of rotation and extends along a direction transverse to the axis of rotation. A movable handle is mounted on the housing so as to be opened and closed with respect to the grip.

The movable handle has a force applying portion to which a manipulating force for opening or closing of the movable handle with respect to the grip is applicable. The force applying portion is positioned on a side of the axis of rotation where the grip is positioned and on the distal-end side with respect to the grip. The housing is held by a hand of a user while palm and thumb of the hand are held against the grip from the proximal-end side and the third finger and/or the little finger of the hand applies the manipulating force to the force applying portion. The index finger applies a manipulating force for rotating the rotary member to the ridge. The cavity of the housing is positioned in a range in which the index finger of the hand can be placed therein while the hand is holding the housing. While the housing is held by the hand and the index finger of the hand is being placed in the cavity, the index finger extends obliquely to the axis of rotation as viewed from one side in a direction that is traverse to the axis of rotation and also to the direction along which the grip extends.

While the embodiments and the modifications have been described hereinbefore, the present disclosure is not limited to the embodiments and the modifications. Various changes and modifications may be made therein without departing from the scope of the disclosure.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A treatment tool comprising:
a housing;
an elongated member configured to be coupled to the housing and being capable of rotating around an axis of rotation with respect to the housing;
an end effector being disposed on a distal-end portion of the elongated member; and
a rotary member configured to be coupled to the elongated member and the housing, the rotary member includes at least one ridge and one cavity, the ridge projecting toward an outer circumferential side and extending from a proximal-end side toward a distal-end side on the rotary member and the cavity being formed on the distal-end side from the ridge on the rotary member and wherein the rotary member being capable of rotating in unison with the elongated member and the end effector around the axis of rotation.

2. The treatment tool of claim 1,
wherein the rotary member includes:
a first slanted surface extending from a distal-end portion of the at least one ridge toward the distal-end side and slanted toward an inner circumferential side of the rotary member gradually from the proximal-end side toward the distal-end side; and
a second slanted surface contiguous to a distal-end side of the first slanted surface and extending toward the distal-end side up to the at least one cavity, the second slanted surface being inclined at a larger tilt angle than the first slanted surface toward the inner circumferential side gradually from the proximal-end side toward the distal-end side of the rotary member.

3. The treatment tool of claim 1,
wherein the rotary member includes a face surface that defines a base of the at least one ridge; and
the at least one cavity is concave toward an inner circumferential side with respect to the face surface.

4. The treatment tool of claim 1,
wherein the at least one cavity is shaped so as to correspond to a shape of an index finger.

5. The treatment tool of claim 1,
wherein the housing includes a first confronting face that confronts the rotary member; and
the rotary member has a second confronting face that confronts the first confronting face, the second confronting face abutting against the first confronting face when a force is applied to the rotary member toward the proximal-end side or in a width-wise direction of the housing.

6. The treatment tool of claim 1,
wherein the at least one ridge includes a plurality of ridges spaced apart on the rotary member around the axis of rotation, and the at least one cavity includes a plurality of cavities spaced apart on the rotary member around the axis of rotation; and
each of the plurality of the cavities is contiguous to the distal-end side of a corresponding one of the plurality of the ridges.

7. The treatment tool of claim 1, further comprising:
a grip configured to be disposed in a region of the housing spaced apart from the axis of rotation and extending along a direction transverse to the axis of rotation; and
a movable handle being mounted on the housing so as to be opened and closed with respect to the grip, the movable handle having a force applying portion to which a manipulating force for opening or closing of the movable handle with respect to the grip is applicable, the force applying portion being positioned on a side of the axis of rotation where the grip is positioned, and on the distal-end side with respect to the grip.

8. The treatment tool of claim 7,
wherein the housing is being held by a hand of a user while palm and thumb of the hand are held against the grip from the proximal-end side and the third finger and/or the little finger of the hand applies the manipulating force to the force applying portion, and the index finger applies a manipulating force for rotating the rotary member to the ridge.

9. The treatment tool of claim 8,
wherein the cavity of the rotary member is positioned in a range in which the index finger of the hand can be placed therein while the hand is holding the housing.

10. The treatment tool of claim 9,
wherein the ridge includes a plurality of ridges spaced apart on the rotary member around the axis of rotation, and the cavity includes a plurality of cavities spaced apart on the rotary member around the axis of rotation;
each of the cavities is contiguous to the distal-end side of a corresponding one of the ridges and
when the user is holding the housing while a fingertip portion of the index finger is placed in one of the cavities, the distal interphalangeal joint of the index finger or a nearby portion thereof is held against one of the ridges and the cavity that is contiguous to the distal-end side of the ridge against which the distal interphalangeal joint of the index finger or the nearby portion thereof is held being disposed adjacent to the cavity.

11. The treatment tool of claim 8,
wherein while the housing is being held by the hand and the index finger of the hand is being placed in the cavity, the index finger extends obliquely to the axis of rotation as viewed from one side in a direction that is traverse to the axis of rotation and also to the direction along which the grip extends.

12. The treatment tool of claim 8,
wherein while the housing is being held by the hand and the index finger of the hand is being placed in the cavity, the index finger is bent toward an inner circumferential side of the rotary member at the distal interphalangeal joint of the index finger.

13. The treatment tool of claim 1,
wherein the end effector is capable of being bended and/or curved with respect to the elongate member.

14. The treatment tool of claim 1, wherein the elongated member is defined by a sheath.

15. The treatment tool of claim 1, wherein the elongated member is defined by a shaft.

16. A treatment tool comprising:
a housing;
a sheath configured to be coupled to the housing and being capable of rotating around an axis of rotation with respect to the housing;
an end effector being disposed on a distal-end portion of the sheath; and
a rotary member configured to be coupled to the sheath and the housing and being capable of rotating in unison with the sheath and the end effector around the axis of the rotation, the rotary member having at least one ridge and at least one cavity and wherein the cavity being disposed on a distal-end side of the ridge.

17. The treatment tool of claim 16, wherein
the ridge has a first surface,
the cavity has a second surface contiguous to a distal-end side of the first surface, and
the second surface is inclined at a larger tilt angle than the first surface.

18. A treatment tool comprising:
a housing including at least one cavity;
a shaft configured to be coupled to the housing and being capable of rotating around an axis of rotation with respect to the housing;
an end effector being disposed on a distal-end portion of the shaft; and
a rotary member configured to be coupled to the shaft and the housing, the rotary member includes at least one ridge, the ridge projecting toward an outer circumferential side and extending from a proximal-end side toward a distal-end side on the rotary member and being formed on the proximal-end side from the cavity and wherein the rotary member being capable of rotating in unison with the shaft and the end effector around the axis of rotation.

19. The treatment tool of claim 16, further comprising:
a grip configured to be disposed in a region of the housing spaced apart from the axis of rotation and extending along a direction transverse to the axis of rotation; and
a movable handle being mounted on the housing so as to be opened and closed with respect to the grip, the movable handle having a force applying portion to which a manipulating force for opening or closing of the movable handle with respect to the grip is applicable, the force applying portion being positioned on a side of the axis of rotation where the grip is positioned, and on the distal-end side with respect to the grip.

20. The treatment tool of claim 17,
wherein the housing is being held by a hand of a user while palm and thumb of the hand are held against the grip from the proximal-end side and the third finger and/or the little finger of the hand applies the manipulating force to the force applying portion, and the index finger applies a manipulating force for rotating the rotary member to the ridge.
